(19) United States
(12) Patent Application Publication
(10) Pub. No.: US 2012/0122089 A1
(43) Pub. Date: May 17, 2012

(54) Gene Marker For Evaluating Genetic Potential For Marbling In Bovine Individual And Method For Evaluating Genetic Potential For Marbling Using The Same

(75) Inventors: Akiko TAKASUGA, Fukushima (JP); Toshio WATANABE, Fukushima (JP); Kou YOKOUCHI, Fukushima (JP); Ken TATSUDA, Hyogo (JP)

(73) Assignee: JAPAN LIVESTOCK TECHNOLOGY ASSOCIATION, Tokyo (JP)

(21) Appl. No.: 13/049,411

(22) Filed: Mar. 16, 2011

(30) Foreign Application Priority Data
Nov. 15, 2010 (JP) 2010-255051

(51) Int. Cl. C12Q 1/68; C07H 21/04

(52) U.S. Cl. 435/6.11; 536/23.5

(57) ABSTRACT

The present invention provides markers for evaluating a genetic potential for marbling of a bovine individual as well as methods for evaluating genetic potential for marbling using the markers.

### CROSS REFERENCE TO RELATED APPLICATION

This application claims the benefit of priority from Japanese Patent Application No. 2010-255051 filed Nov. 15, 2010, which is herein incorporated by reference.

### TECHNICAL FIELD

The present invention relates to markers for evaluating a genetic potential for marbling of a bovine individual, as well as methods for evaluating the genetic potential for marbling using the markers.

### BACKGROUND ART

Meat quality and carcass weight of beef cattle are economic traits directly linked with prices. In particular, the marbling, which means a state of meat where fat is accumulated in muscular tissues, is appreciated to be in good quality if the fat is deposited finely and thickly like marble, and it affects various meat qualities such as tenderness. In order to evaluate the genetic potential for the meat quality and carcass weight and utilize the evaluation to improve cattle, methods based on breeding values etc. have been developed and used.

The meat quality and carcass weight are considered to be quantitative traits in which multiple genes are involved. If a gene or a genomic region, or a quantitative trait locus (QTL), having not a little influence on meat quality or carcass weight, can be identified and it becomes possible to select a superior genotype, it can be utilized to improve cattle breeding.

It has been reported with a QTL mapping study using a paternal half-sib family of a Japanese Black (Wagyu) bull (Mizoguchi et al. (2006) Animal Genetics 37, 51-54) that a genomic region involved in marbling is present on bovine chromosome 4. Later, in another Japanese Black bull's half-sib family, a QTL for marbling was also identified in the same region on chromosome 4, and this QTL was shown to be present in a 3.7 Mb region at around 46 cM by a haplotype comparison and association analysis (Yokouchi et al. (2009) Animal Genetics 40, 945-951).

### SUMMARY OF INVENTION

### Technical Problem

However, since it was not known what kind of genetic information was actually involved in the superior trait, it was impossible to utilize genetic information such as genotypes when evaluating genetic potential for marbling of a bovine individual.

Thus, an object of the present invention is to provide a marker to evaluate a genetic potential for marbling of a bovine individual as well as a method for evaluating the genetic potential for marbling using the marker.

### Solution to Problem

By analyzing in detail the genomic region on bovine chromosome 4, which is involved in marbling, the inventors of the present invention discovered that among the SNPs (Single Nucleotide Polymorphisms) located in the upstream region of bovine pantophysin gene, an SNP at S1 site (also referred as SNP27) is related to marbling, and thus DNA that contains the S1 site where the nucleotide is A is useful as a gene marker for marbling. Further, based on the discoveries that each of the SNPs of SNP29, SNP30, ARS-BFGL-NGS-20161, BTB-00183730 and BTA-70505-no-rs was in linkage disequilibrium with the SNP at the S1 site and that the expression of the pantophysin gene was relatively higher in the intramuscular adipose tissue of bovine individuals in which the nucleotide at the S1 site was A, the inventors concluded that the S1 site is responsible for the marbling QTL, and thus accomplished the present invention.

As used herein, the “S1 site” represents the position that corresponds to the 49100585^{th} nucleotide on chromosome 4 of the bovine genomic sequence (Btau4.0) and corresponds to the 718^{th} nucleotide upstream of exon 1 of the bovine pantophysin gene, as set forth in SEQ ID NO:1.

SNP29 herein represents the SNP at the position that corresponds to the 49101189^{th} nucleotide on chromosome 4 of the bovine genomic sequence (Btau4.0) and corresponds to the 1322^{th} nucleotide upstream of exon 1 of the bovine pantophysin gene, as set forth in SEQ ID NO:1.

SNP30 herein represents the SNP at the position that corresponds to the 49101317^{th} nucleotide on chromosome 4 of the bovine genomic sequence (Btau4.0) and corresponds to the 1450^{th} nucleotideupstream of exon 1 of the bovine pantophysin gene, as set forth in SEQ ID NO:1.

ARS-BFGL-NGS-20161 (NCBI Assay ID: ss86290215) herein represents the SNP at the position that corresponds to the 49036518^{th} nucleotide on chromosome 4 of the bovine genomic sequence (Btau4.0) and corresponds to the 27348^{th} nucleotide downstream of exon 7 of the bovine pantophysin gene, as set forth in SEQ ID NO:2.

BTB-00183730 (NCBI Reference SNP: rs43398415) herein represents the SNP at the SNP at the position that corresponds to the 49109714^{th} nucleotide on chromosome 4 of the bovine genomic sequence (Btau4.0) and corresponds to the 9847^{th} nucleotide upstream of exon 1 of the bovine pantophysin gene, as set forth in SEQ ID NO:3.

BTA-70505-no-rs (NCBI Reference SNP: rs41602690) herein represents the SNP at the position corresponding to the 49147499^{th} nucleotide on chromosome 4 of the bovine genomic sequence (Btau4.0) and corresponds to the 47632^{th} nucleotide upstream of exon 1 of the bovine pantophysin gene, as set forth in SEQ ID NO:4.

As used herein, the “sense strand” of the pantophysin gene refers to the one of the two strands, which encodes a pantophysin protein. Therefore, the bovine genomic sequence of Btau4.0 refers to the anti-sense strand. In the present specification, SNPs are represented by a nucleotide in the sense strand, unless otherwise described.

As used herein, the N^{th} position upstream of exon 1 refers to the position counted toward upstream direction from a nucleotide flanking on the upstream end of exon 1.

As used herein, the N^{th} position downstream of exon 7 refers to the position counted toward downstream direction from a nucleotide flanking on the downstream end of exon 7.

SNPs are herein defined by the number of the position on chromosome 4 of the bovine genomic sequence (Btau4.0) as well as the number counted toward upstream direction out of exon 1 of pantophysin gene, or the number counted toward downstream direction out of exon 7 of pantophysin gene, as set forth in SEQ ID NOs:1 to 4. In individual cases, SNPs may be located at the position that corresponds to the abovementioned position, and their actual position number may be different due to deletion and/or insertion of nucleotide(s) in their specific genomic DNA.

An embodiment of the present invention is an isolated DNA including a nucleotide corresponding to the nucleotide at the 718^{th} position upstream of exon 1 of the bovine pantophysin gene as set forth in SEQ ID NO:1, wherein the nucleotide included in the DNA is A.

Another embodiment of the present invention is an isolated DNA including the nucleotide corresponding to the nucleotide at the 1322^{th} position upstream of exon 1 of the bovine pantophysin gene as set forth in SEQ ID NO:1, wherein the nucleotide included in the DNA is G.

Further embodiment of the present invention is an isolated DNA including the nucleotide corresponding to the nucleotide at the 1450^{th} position upstream of exon 1 of the bovine pantophysin gene as set forth in SEQ ID NO:1, wherein the nucleotide included in the DNA is T.

A marker according to the present invention is a marker for evaluating a genetic potential for increasing the degree of marbling of a bovine individual, and is selected from the group consisting of DNAs, each of which includes the nucleotide corresponding to either of the nucleotides at: the 718^{th}, 1322^{th} and 1450^{th} positions upstream of exon 1 of the bovine pantophysin gene as set forth in SEQ ID NO:1; the 9847^{th} position upstream of exon 1 of the bovine pantophysin gene as set forth in SEQ ID NO:3; the 47632^{th} position upstream of exon 1 of the bovine pantophysin gene as set forth in SEQ ID NO:4; and the 27348^{th} position downstream of exon 7 of the bovine pantophysin gene as set forth in SEQ ID NO:2.

A method according to the present invention for evaluating a genetic potential for increasing the degree of marbling of a bovine individual includes the step of judging whether or not the nucleotide corresponding to the nucleotide at the 718^{th} position upstream of exon 1 of the bovine pantophysin gene as set forth in SEQ ID NO:1 is A in at least one of alleles in DNA isolated from the bovine individual.

A method according to the present invention for evaluating a genetic potential for increasing the degree of marbling of a bovine individual includes the step of judging whether or not the nucleotide corresponding to the 1322^{th} nucleotide upstream of exon 1 of the bovine pantophysin gene as set forth in SEQ ID NO:1 is G in at least one of alleles in DNA isolated from the bovine individual.

A method according to the present invention for evaluating a genetic potential for increasing the degree of marbling of a bovine individual includes the step of judging whether or not the nucleotide corresponding to the 1450^{th} nucleotide upstream of exon 1 of the bovine pantophysin gene as set forth in SEQ ID NO:1 is T in at least one of alleles in DNA isolated from the bovine individual.

A method according to the present invention for evaluating a genetic potential for increasing the degree of marbling of a bovine individual includes the step of judging whether or not the nucleotide corresponding to the 27348^{th} nucleotide downstream of exon 7 of the bovine pantophysin gene as set forth in SEQ ID NO:2 is A in at least one of alleles in DNA isolated from the bovine individual.

A method according to the present invention for evaluating a genetic potential for increasing the degree of marbling of a bovine individual includes the step of judging whether or not the nucleotide corresponding to the 9847^{th} nucleotide upstream of exon 1 of the bovine pantophysin gene as set forth in SEQ ID NO:3 is C in at least one of alleles in DNA isolated from the bovine individual.

A method according to the present invention for evaluating a genetic potential for increasing the degree of marbling of a bovine individual includes the step of judging whether or not a nucleotide corresponding to the 47632^{th} nucleotide upstream of exon 1 of the bovine pantophysin gene as set forth in SEQ ID NO:4 is T in at least one of alleles in DNA isolated from the bovine individual.

Preferably, any of the abovementioned evaluation methods according to the present invention may further include the step of judging that the genetic potential for increasing the degree of marbling of the bovine individual is higher than the genetic potential of another bovine individual whose nucleotide corresponding to the 718^{th} nucleotide upstream of exon 1 of the bovine pantophysin gene as set forth in SEQ ID NO:1 is G.

A method according to the present invention for selecting a bovine individual having a high genetic potential for increasing the degree of marbling includes the step of evaluating the genetic potential of the bovine individual by any one of the abovementioned methods of evaluation. Preferably, this method may further include the step of selecting the bovine individual judged as having the higher genetic potential by the step of evaluation.

### BRIEF DESCRIPTION OF DRAWINGS

FIG. 1

FIG. 1 shows the results of analyses of 26 SNP markers on the bovine chromosome 4 on (A) the correlation between Q haplotypes from each pair of adjacent markers and marbling scores and (B) the linkage disequilibrium between SNPs, using 406 animals in a high-marbling score group (marbling score (BMS) was 9 or higher) and 412 animals in a low-marbling score group (BMS was 4 or lower) taken from 19112 Japanese Black steers in one embodiment of the present invention.

FIG. 2

FIG. 2 is a graph showing the expression levels of four genes, located in a ca. 0.7 Mb region with linkage disequilibrium including SNP20 and SNP21 on the bovine chromosome 4, in the intramuscular adipose tissue of bovine individuals homozygous of the superior haplotype (Q) or the non-superior haplotype (q) in one embodiment of the present invention (N represents the number of individuals).

FIG. 3

FIG. 3 shows the locations of 6 SNPs (SNP27 to SNP32) present in the 2 kb region upstream of exon 1 of the pantophysin gene, as well as a graph showing (A) promoter activities of the 1.8 kb region containing the 6 SNPs and the 0.9 kb region containing only SNP27, having either the superior haplotype (Q) or the non-superior haplotype (q), and (B) promoter activities in the cases where only SNP27 among the 6 SNPs was replaced with a q-type nucleotide (−1.8 k(Q)_S27q/pGL3) in one embodiment of the present invention.

FIG. 4

FIG. 4 is a graph showing the degrees of linkage disequilibrium between two SNPs selected from SNP27-32 in a population of Japanese Black in one embodiment of the present invention.

FIG. 5

FIG. 5 is a graph showing the degrees of linkage disequilibrium between SNP27 and other known SNPs in a population of Japanese Black in one embodiment of the present invention.

### DESCRIPTION OF EMBODIMENTS

Embodiments of the present invention accomplished based on the above-described findings are hereinafter described in detail by giving Examples.

Unless otherwise explained, methods described in standard sets of protocols such as J. Sambrook and E. F. Fritsch & T. Maniatis (Ed.), “Molecular Cloning, a Laboratory Manual (3rd edition), Cold Spring Harbor Press and Cold Spring Harbor, N.Y. (2001); and F. M. Ausubel, R. Brent, R. E. Kingston, D. D. Moore, J. G. Seidman, J. A. Smith, and K. Struhl (Ed.), “Current Protocols in Molecular Biology,” John Wiley & Sons Ltd., or alternatively, modified/changed methods from these are used. When using commercial reagent kits and measuring apparatus, unless otherwise explained, attached protocols to them are used.

The objective, characteristics, and advantages of the present invention as well as the idea thereof will be apparent to those skilled in the art from the descriptions given herein. It is to be understood that the embodiments and specific examples of the invention described hereinbelow are to be taken as preferred examples of the present invention. These descriptions are for illustrative and explanatory purposes only and are not intended to restrict the invention to these embodiments or examples. It is further apparent to those skilled in the art that various changes and modifications may be made based on the descriptions given herein within the intent and scope of the present invention disclosed herein.

=SNPs Around the Bovine Pantophysin Gene=

The nucleotide on the sense DNA strand of the bovine genomic sequence (Btau4.0) at the site corresponding to the S1 site is C, and thus the nucleotide at the S1 site in the sense DNA strand of the upstream region of exon 1 of bovine pantophysin gene is G. As illustrated in Examples, however, in cases where the nucleotide at the S1 site is A, the pantophysin gene is expressed at a higher level, and the degree of marbling increases. Accordingly, determination of the nucleotide at the S1 site among the SNPs in the upstream region of exon 1 of the bovine pantophysin gene makes it possible to evaluate or estimate the genetic potential for marbling. As used herein, the upstream region of exon 1 of the bovine pantophysin gene refers to a region located outside of exon 1 and on the upstream side of exon 1 that is included in the transcribed regions of the bovine pantophysin gene.

Examples of the SNPs located in the upstream region of exon 1 of the pantophysin gene, which are in linkage disequilibrium with the SNP at the S1 site, include SNP29, SNP30, BTB-00183730 and BTA-70505-no-rs. On the other hand, an example of the SNPs located in the downstream region of exon 7 of the pantophysin gene, which are in linkage disequilibrium with the SNP at the S1 site includes ARS-BFGL-NGS-20161. As used herein, the downstream region of exon 7 of the bovine pantophysin gene refers to a region located outside of exon 7 and on the downstream side of exon 7 that is included in the transcribed regions of the bovine pantophysin gene.

Specifically, in cases where the nucleotide at the S1 site is G, the nucleotide at SNP29 is C, the nucleotide at SNP30 is A, the nucleotide at ARS-BFGL-NGS-20161 is G, the nucleotide at BTB-00183730 is T and the nucleotide at BTA-70505-no-rs is C. Meanwhile, in cases where the nucleotide at the S1 site is A, the nucleotide at SNP29 is G, the nucleotide at SNP30 is T, the nucleotide at ARS-BFGL-NGS-20161 is A, the nucleotide at BTB-00183730 is C and the nucleotide at BTA-70505-no-rs is T. Accordingly, by determining the nucleotide at SNP29, SNP30, BTB-00183730 or BTA-70505-no-rs among the SNPs in the upstream region of exon 1 of the bovine pantophysin gene or at ARS-BFGL-NGS-20161 in the downstream region of exon 7 of the bovine pantophysin gene and examining if it is mutated or not, the genetic potential for marbling can be evaluated or estimated, similarly to the cases using the S1 site.

=Marker=

The marker for evaluating the degree of marbling of a bovine individual according to the present invention may be an isolated bovine genomic DNA including the S1 site in the bovine genomic sequence (Btau4.0). When the nucleotide at the S1 site is mutated, the nucleotides at the SNPs in linkage disequilibrium with the SNP at the S1 site are also mutated with high probability. Therefore, the marker may alternatively be an isolated bovine genomic DNA including any one of the SNPs in linkage disequilibrium with the SNP at the S1 site, and examples of such a bovine genomic DNA include the genomic DNAs including any of SNP29, SNP30, ARS-BFGL-NGS-20161, BTB-00183730 and BTA-70505-no-rs.

When the marker is a DNA, a nucleotide at the SNP site may be determined in order to detect the SNP. Specifically, the nucleotide sequence may be directly determined; or PCR or RFLPs may be used. The method for the detection is not particularly limited. When the SNP is directly detected, the genomic DNA whose sequence is to be determined is not required to contain the entire pantophysin gene but is sufficient as long as it contains the nucleotide of the SNP to be determined and the nucleotide can be determined.

=Method for Evaluating a Genetic Potential for Marbling=

It can be examined by determining the nucleotide at the S1 site whether or not the nucleotide at the S1 site is A in at least one of alleles of DNA isolated from a bovine individual. For example, the nucleotide at the S1 site may be determined by any of conventional methods following extraction of genomic DNA from bovine cells.

When the SNP at the S1 site is heterozygous (G/A) or homozygous (A/A) in a bovine individual, then the genetic potential for increasing the degree of marbling of the bovine may be judged to be higher than that of a bovine individual homozygous (G/G) at the SNP. When the SNP is homozygous (A/A), then the genetic potential for increasing the degree of marbling of the bovine individual may be judged to be even higher than a bovine individual being heterozygous (G/A) at the SNP.

While the SNP at the S1 site can be determined most accurately by examining the nucleotide at the S1 site, the SNP at the S1 site can be also estimated by examining any of the SNPs in linkage disequilibrium with the S1 site. Examples of the SNPs in linkage disequilibrium with the S1 site include SNP29, SNP30, ARS-BFGL-NGS-20161, BTB-00183730 and BTA-70505-no-rs. Specifically, it may be determined, in DNA isolated from a bovine individual, whether or not the nucleotide at either of SNP29, SNP30, ARS-BFGL-NGS-20161, BTB-00183730 or BTA-70505-no-rs is G, T, A, C or T in at least one allele, respectively. It is not necessary to determine all of these SNPs, but rather at least one of them should be determined. For better accuracy in the estimation of the SNP at the S1 site, however, preferably two or more of the SNPs at SNP29, SNP30, ARS-BFGL-NGS-20161, BTB-00183730 and BTA-70505-no-rs may be determined; more preferably any three of the SNPs are determined; even more preferably any four of the SNPs are determined; and most preferably all five SNPs are determined.

When SNP29 is heterozygous (C/G) or homozygous (G/G) in a bovine individual, then SNP at the S1 site may be estimated to be heterozygous (G/A) or homozygous (A/A), respectively, and thus the genetic potential for increasing the degree of marbling of the bovine individual may be judged to be higher than the potential of an individual being homozygous (C/C) at SNP29 or an individual being homozygous (G/G) at the S1 site. When SNP29 is homozygous (G/G), then the genetic potential for increasing the degree of marbling of the bovine individual may be judged to be even higher than the potential of an individual being heterozygous (C/G) at SNP29 or an individual being heterozygous (G/A) at the S1 site.

When SNP30 is heterozygous (A/T) or homozygous (T/T) in a bovine individual, then the SNP at the S1 site may be estimated to be heterozygous (G/A) or homozygous (A/A), respectively, and thus the genetic potential for increasing the degree of marbling of the bovine individual may be judged to be higher than the potential of an individual being homozygous (A/A) at SNP30 or an individual being homozygous (G/G at the S1 site. When SNP30 is homozygous (T/T), then the genetic potential for increasing the degree of marbling of the bovine individual may be judged to be even higher than the potential of an individual being heterozygous (A/T) at SNP30 or an individual being heterozygous (G/A) at the S1 site.

When ARS-BFGL-NGS-20161 is heterozygous (G/A) or homozygous (A/A) in a bovine individual, then the SNP at the S1 site may be estimated to be heterozygous (G/A) or homozygous (A/A), and thus the genetic potential for increasing the degree of marbling of the bovine individual may be judged to be higher than the potential of an individual being homozygous (G/G) at ARS-BFGL-NGS-20161 or an individual being homozygous (G/G) at the S1 site. When ARS-BFGL-NGS-20161 is homozygous (A/A), then the genetic potential for increasing the degree of marbling of the bovine individual may be judged to be even higher than the potential of an individual being heterozygous (G/A) at ARS-BFGL-NGS-20161 or an individual being heterozygous (G/A) at the S1 site.

When BTB-00183730 is heterozygous (T/C) or homozygous (C/C) in a bovine individual, then the SNP at the S1 site may be estimated to be heterozygous (G/A) or homozygous (A/A), respectively, and thus the genetic potential for increasing the degree of marbling of the bovine individual may be judged to be higher than the potential of an individual being homozygous (T/T) at BTB-00183730 or an individual being homozygous (G/G) at the S1 site. When BTB-00183730 is homozygous (C/C), then the genetic potential for increase the degree of marbling of the bovine individual may be judged to be even higher than the potential of an individual being heterozygous (T/C) at BTB-00183730 or an individual being heterozygous (G/A) at the S1 site.

When BTA-70505-no-rs is heterozygous (C/T) or homozygous (T/T) in a bovine individual, then the SNP at the S1 site may be estimated to be heterozygous (G/A) or homozygous (A/A), respectively, and thus the genetic potential for increasing the degree of marbling of the bovine individual may be judged to be higher than the potential of an individual being homozygous (C/C) at BTA-70505-no-rs or an individual being homozygous (G/G) at the S1 site. When BTA-70505-no-rs is homozygous (T/T), then the genetic potential for increasing the degree of marbling of the bovine individual may be judged to be even higher than the potential of an individual being heterozygous (C/T) at BTA-70505-no-rs or an individual being heterozygous (G/A) at the S1 site.

Further, any of the abovementioned methods for evaluation may be employed to select an individual having a high genetic potential for increasing the degree of marbling among many bovine individuals. To this end, the nucleotides at the S1 site in the upstream region of exon 1 of the pantophysin gene may be determined for each of the bovine individuals, and an individual where the nucleotide at the S1 site is A in one or both of alleles may be selected. Alternatively, the nucleotides at any of the SNPs of SNP29, SNP30, ARS-BFGL-NGS-20161, BTB-00183730 and BTA-70505-no-rs may be determined for each of the bovine individuals, and an individual where the nucleotide at SNP29 is G, the nucleotide at SNP30 is T, the nucleotide at ARS-BFGL-NGS-20161 is A, the nucleotide at BTB-00183730 is C or the nucleotide at BTA-70505-no-rs is T, in one or both of alleles, may be selected.

Since the pantophysin gene as well as the upstream region of exon 1 and the downstream region of exon 7 of the pantophysin gene are highly conserved among various bovine breeds, the breed to be subjected to the present invention is not particularly limited, and may include Japanese Black, Japanese Brown, Holstein and others.

### Examples

Hereinafter the present invention will be explained in more detail with reference to Examples and drawings.

### 1. Methods for Extracting DNA and Genotyping Microsatellites and SNPs

Genomic DNA was extracted from semen, perinephric fat or blood by a conventional method. Each of the relevant genomic regions was amplified by the PCR method using specific primers for amplification of the genomic fragments of interest. Microsatellites were genotyped by PCR amplification using forward and fluorescent-labeled reverse primers, followed by electrophoresis using ABI 3730 DNA analyzer (Applied Biosystems) and analysis using GENESCAN and GeneMapper software (Applied Biosystems). Detection and genotyping of SNPs were conducted by direct sequencing of the PCR products using Big Dye Terminator v.3.1 Cycle Sequencing Kit (Applied Biosystems). The primers used for each of the SNPs (SEQ ID NOs:5 to 66) were shown in Table 1.

TABLE 1

| SNP | Forward primer | Reverse primer |
|---|---|---|
| 1 | CCTTGAATTGGAGCAGCA | GCCATAGGTAGTGATGCAAGC |
| 2 | GCAGTAGCCTGTTTTCTGCTT | TATATGCACGTTGGCCACTC |
| 3 | AAGAATTCCCATCCCTGAGC | CAACTTTCTGCATGGGAGAC |
| 4 | GGTGGATCAGCATCCTGTAGA | CTTCTCTGGCCACCCCTATT |
| 5 | TCCCTCAGACACTCCCTTGT | AGAGGCCACCATTGAGAGAA |
| 6 | TCCTCAGTCTGGGAAAGGAA | GCACACCCAATGTTAAGCTG |
| 7 | CATTCTTCCCCTCAACCATC | GGACACTTTTTCTTGCCTGA |
| 8 | ATGTGCAGGGAAATGAGCAC | CGCCTCCAAAACTGAATGAG |
| 9 | AGAGAGCCTGGGAGTTGTCA | TATGGGACCTGGATGCATTG |
| 10 | TTTGAATGAGCATTTCTCACG | CTCAGGCACGTGGTATATGAA |
| 11 | TCCAGTTCTCCCATGAAACC | AAGAAGAGCCCTAACGCAGA |
| 12 | TTGGAAGGGCTATAGTTGCTG | GGAGGCAGGTGGAATGTTTA |
| 13 | CAGTGGCAAAAGCTCAGAAC | AATGTGGCTGAGAAGCCTTG |
| 14 | GACCTCATTCAGGAGGCAAA | GGCAGGACCCTTGAAATTG |
| 15 | TTTGAATTCCCACCTTGAAC | TTCCCAAAAGTCCTTGCATC |
| 16 | TCAGACTCAGCCAACAGCAC | ACTGCTCCACAGCAAAAACC |
| 17 | GGTGAACAGCAGGGAAGTTT | TGAGCTGTGCAGTGTCCAAT |
| 18 | CTGGAAGTGTCCGGGAAAT | TGCACTCCTAGGTGAAGACG |
| 19 | CCCTAAACCCTGTCACCAGA | TCAGGAATGGACCAGAGGAC |
| 20 | TCTCCAGAACATGCATCCTG | GGTGGATCCTGGAATTGCTA |
| 21 | TTAACCAGCAAGGAGGCAAC | CGGAGCCTACCCCAAATAGT |
| 22 | TGGAGGTTTTAAGGGCAAAA | CAGATAGGCACTGGTCCACA |
| 23 | CTTTTGCCCCACATGATTTT | TGGGTTTAGACGCAGCTCTT |
| 24 | AAACCAGGTTCGAGTTGCAC | ACGGTCGAAAATCCAGTCAG |
| 25 | TCTCTCCTCCTTCCCATTCA | CTTCTGCTGGCCACTCTTTC |
| 26 | CAGTGGCTGCACTAATCCAA | CCTCAGAACTGCCCTGAAAG |
| 27 | CAAAGGCCAGCATTTCATCT | CCACCTCTCCTACCCTGTCA |
| 28 | GCAGGGATTGAAGAATGGAA | TGGCCTTTGGAGTGTATGGT |
| 29 | GGAAGCCAGTACGGAGATCA | CCGGACCACAGAGACAATCT |
| 30 | common with SNP29 | common with SNP29 |
| 31 | AATTGGGGAGGGGAATACAG | TCTCCGTACTGGCTTCCTTG |
| 32 | CTGAGAAAGGGCCATGTCAA | CTGGCTTGGGCTTCATATTC |

### 2. Methods for Evaluation of Marbling

Evaluation of marbling is systematically conducted by certified graders on the basis of the Beef Marbling Standard (BMS) scores at each slaughterhouse. The BMS scores were used as phenotypic values for marbling.

### 3. Statistical Analysis of SNPs in Relation to Marbling Score

This Example shows that the nucleotide at the S1 site in the upstream region of exon 1 of the pantophysin gene and the marbling score are strongly correlated.

The inventors of the present invention have reported that the marbling QTL on bovine chromosome 4 having been detected by the QTL mapping studies using two Japanese Black sires (A and B) is located in a 3.7 Mb region at around 46 cM (Yokouchi et al. (2009) Animal Genetics 40, 945-951). Since the sires A and B had common superior (Q) and non-superior (q) haplotypes in this region, SNPs different between Q and q haplotypes were searched for to narrow the region further, and the 26 SNP markers thus obtained (Table 2) were subjected to analyses of relationship between the frequency of Q allele and the marbling.

TABLE 2

| | Position | Nucleotide | Q/q | |
|---|---|---|---|---|
| SNP | (Btau4.0) | (Antisence strand) | (Antisence strand) | Hardy-W P |
| 1 | 45,938,113 | C | A/C | 0.89042 |
| 2 | 46,042,897 | G | G/A | 0.29337 |
| 3 | 46,149,277 | G | A/G | 0.6987 |
| 4 | 46,245,308 | T | C/T | 0.60153 |
| 5 | 46,355,050 | G | G/A | 0.05951 |
| 6 | 46,489,908 | T | T/C | 0.29152 |
| 7 | 46,599,716 | G | G/A | 0.56418 |
| 8 | 47,035,818 | T | G/T | 0.999 |
| 9 | 47,430,975 | G | A/G | 0.93256 |
| 10 | 47,316,021 | T | A/T | 0.96922 |
| 11 | 47,571,983 | C | A/C | 0.33028 |
| 12 | 47,717,378 | G | G/A | 0.44066 |
| 13 | 47,822,367 | T | C/T | 0.71932 |
| 14 | 47,916,406 | CTGA | TTG/CTGA | 0.20669 |
| 15 | 48,179,960 | G | G/T | 0.29168 |
| 16 | 48,239,249 | A | A/G | 0.60622 |
| 17 | 48,339,135 | T | C/T | 0.48889 |
| 18 | 48,465,214 | C | T/C | 0.40546 |
| 19 | 48,751,701 | G | G/A | 0.30945 |
| 20 | 48,839,441 | C | C/T | 0.28456 |
| 21 | 48,966,107 | G | A/G | 0.23583 |
| 22 | 49,073,999 | C | C/T | 0.49637 |
| 23 | 49,102,772 | G | G/C | 0.15065 |
| 24 | 49,268,950 | T | T/deletion | 0.22347 |
| 25 | 49,383,227 | G | A/G | 0.40532 |
| 26 | 49,482,525 | A | G/A | 0.64725 |

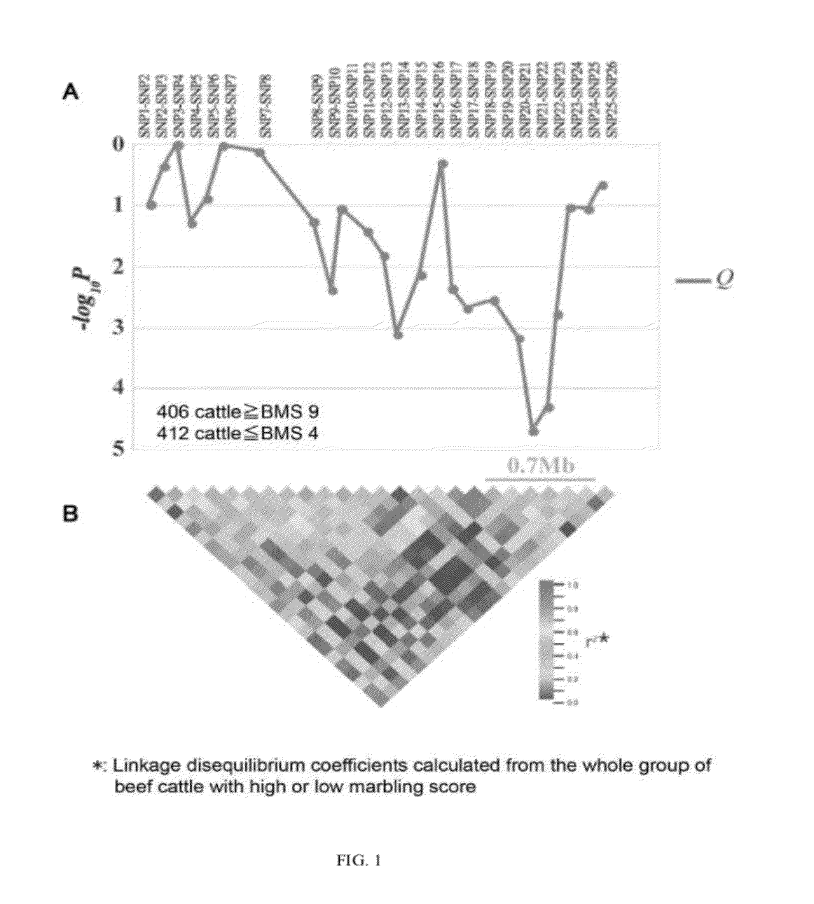

Among 19,112 Japanese Black steers, 406 animals (up to 5 offspring from the same sire) in the high-marbling score group (BMS≧9; top 9.6%) and 412 animals (up to 5 offspring from the same sire) in the low-marbling score group (BMS≦4; bottom 12.2%) were genotyped by using these SNP markers, and haplotype frequencies between two adjacent SNPs were estimated by using ARLEQUIN program (http://lgb.unige.ch/arlequin/). Fisher's exact test was performed to confirm that these SNPs were not deviated from the Hardy-Weinberg equilibrium (p>0.05) required for the algorithm of the haplotype frequency estimation. Correlations between the estimated Q haplotype frequencies and the marbling were tested by Fisher's exact test using 2×2 tables, and resultant p values (see “p value” in Table 3) were plotted as shown in FIG. 1A. As used herein, a “Q haplotype” refers to the common haplotype which was detected by the analysis of multiple bovine families and has the effect of improving BMS. The correlation was found to be most strong between SNP20 and SNP21 (SNP20-SNP21 in Table 2: p=2.01×10⁻⁵), and thus the marbling QTL was considered to be located in a ˜0.7 Mb region with linkage disequilibrium (FIG. 1) containing these SNPs. The coefficient of the linkage disequilibrium (r²) between each of the SNPs in a combined group of the high- and low-marbling grade groups is shown in FIG. 1B. The r² was 0.2 or more in most cases between two SNPs from the SNPs located in the 0.7 Mb region, indicating that they are in linkage disequilibrium with each other.

TABLE 3

| Marker pair | p value |
|---|---|
| SNP1-SNP2 | 0.1025790 |
| SNP2-SNP3 | 0.4297630 |
| SNP3-SNP4 | 0.9594590 |
| SNP4-SNP5 | 0.0503697 |
| SNP5-SNP6 | 0.1268780 |
| SNP6-SNP7 | 0.9201870 |
| SNP7-SNP8 | 0.7224350 |
| SNP8-SNP9 | 0.0527875 |
| SNP9-SNP10 | 0.0040743 |
| SNP10-SNP11 | 0.0871830 |
| SNP11-SNP12 | 0.0367863 |
| SNP12-SNP13 | 0.0148054 |
| SNP13-SNP14 | 0.0007645 |
| SNP14-SNP15 | 0.0073195 |
| SNP15-SNP16 | 0.4858350 |
| SNP16-SNP17 | 0.0042543 |
| SNP17-SNP18 | 0.0020353 |
| SNP18-SNP19 | 0.0027829 |
| SNP19-SNP20 | 0.0006641 |
| SNP20-SNP21 | 0.0000201 |
| SNP21-SNP22 | 0.0000488 |
| SNP22-SNP23 | 0.0016298 |
| SNP23-SNP24 | 0.0913426 |
| SNP24-SNP25 | 0.0853304 |
| SNP25-SNP26 | 0.2143650 |

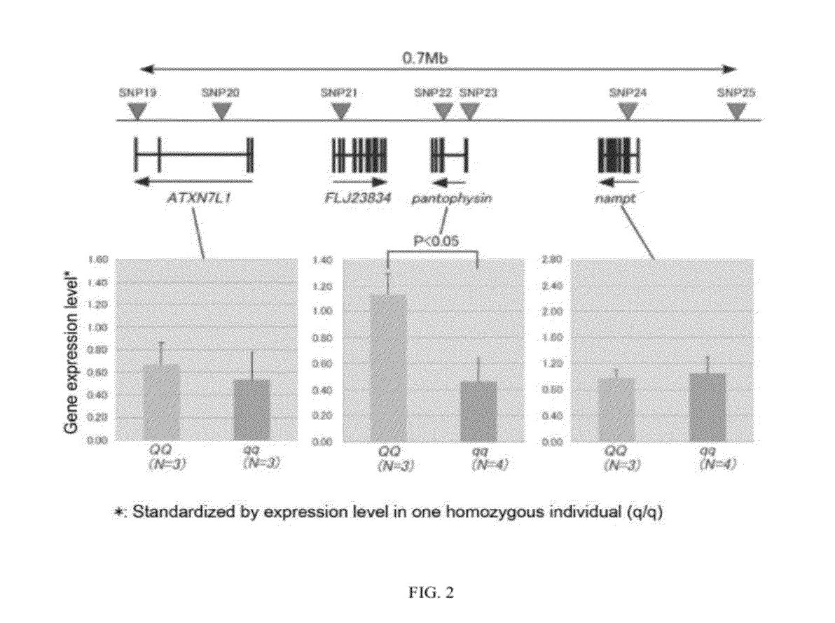

Four genes (ATXN7L1, FLJ23834, pantophysin and nampt) were annotated in the 0.7 Mb orthologous region in human, among which FLJ23834 was considered not to be the responsible gene for the marbling QTL because it contains a nonsense mutation in case of bovine and its expression was not detected in bovine muscle nor adipose tissues. Also for the remaining 3 genes, no mutation involving an amino acid substitution between the Q and q haplotypes was detected. However, when expression levels of these genes in the intramuscular adipose tissue were compared between individuals having the Q allele and individuals having the q allele by using the probes (SEQ ID NOs:67 to 70) and primers (SEQ ID NOs:71 to 78) as shown in Table 4, only the expression of the pantophysin gene was found to be significantly higher in Q-homozygotes than in q-homozygotes (FIG. 2). Thus, a 2 kb region in the 5′ upstream of the pantophysin gene was further analyzed, and 6 SNPs that were different between the Q and q haplotypes were detected (Table 5).

TABLE 4

| | Probe or | |
|---|---|---|
| Gene | primer | Sequence |
| ATXN7L1 | Dual labeled | ACAGGCGCTGCACACCACGAGG |
| | probe | |
| | Forward primer | GGGAGGTTATGAGGCTTAATAAAGAAG |
| | Reverse primer | GGAAGACCTGCGGCTTGAC |
| FLJ23834 | Dual labeled | CTCGGTCTATTCTGCGTGCCACTTGGA |
| | probe | |
| | Forward primer | ACTACTGTCAACAGCTATTTCATGATC |
| | Reverse primer | CATGATGGGATTTTGTCTTAATTCGC |
| Panto- | Dual labeled | CCAGGCTGAAGTGCTCACCAACCACA |
| physin | probe | |
| | Forward primer | CTTCTGCTGTATGTTGGTTACACG |
| | Reverse primer | GGTAGCTGCTTTAATGTCTGTAAGAG |
| Nampt | Dual labeled | CGCTCCTATGCCAGCAGTCTCTTGG |
| | probe | |
| | Forward primer | TTACATGATTTTGGCTACAGAGGAG |
| | Reverse primer | TTCCTTTGAAGTTAACCAAATGAGC |

TABLE 5

| | Position | Nucleotide in | |
|---|---|---|---|
| SNP | (Btau4.0) | sense strand | Q/q |
| 27 | 49,100,585 | G | A/G |
| 28 | 49,101,023 | C | C/T |
| 29 | 49,101,189 | C | G/C |
| 30 | 49,101,317 | A | T/A |
| 31 | 49,101,440 | T | T/A |
| 32 | 49,101,619 | T | T/C |

Further, an effect of these 6 SNPs on the promoter activity was examined by a luciferase assay using bovine clonal intramuscular preadipocytes (BIP; Aso et at (1995) Biochem Biophys Res Commun 213, 369-375) and a luciferase reporter vector (pGL3). First, each of a 1.8 kb (SEQ ID NO:79) and a 0.9 kb DNA fragments (SEQ ID NO:80) of the upstream region of exon 1 of bovine pantophysin gene was inserted into the upstream of the luciferase reporter gene in the vector plasmid. Six nucleotides differ in the 1.8 kb fragment and only one nucleotide for SNP27 differs in the 0.9 kb fragment between the Q and q haplotypes. Specifically, each of the 1.8 kb upstream region of exon 1 and the 0.9 kb upstream region of exon 1 was amplified from bovine genomic DNA by PCR using a pair of primers designed for the both ends of respective regions (the primers for 1.8 kb: SEQ ID NOs:81 and 82; and the primers for 0.9 kb: SEQ ID NOs:82 and 83; shown below), and the PCR products were inserted into the multicloning site of pGL3-Basic (Promega) using DNA Ligation kit (Takara).

(SEQ ID NO: 81)
1.8kb-f: GGGGCTAGCAATTGGGGAGGGGAATACAG
(SEQ ID NO: 82)
1.8kb-r/0.9kb-r: GGGAGATCTCCCCTTCCCTCAAGTCCAG
(SEQ ID NO: 83)
0.9kb-f: GGGGCTAGCCAAAGGCCAGCATTTCATCT

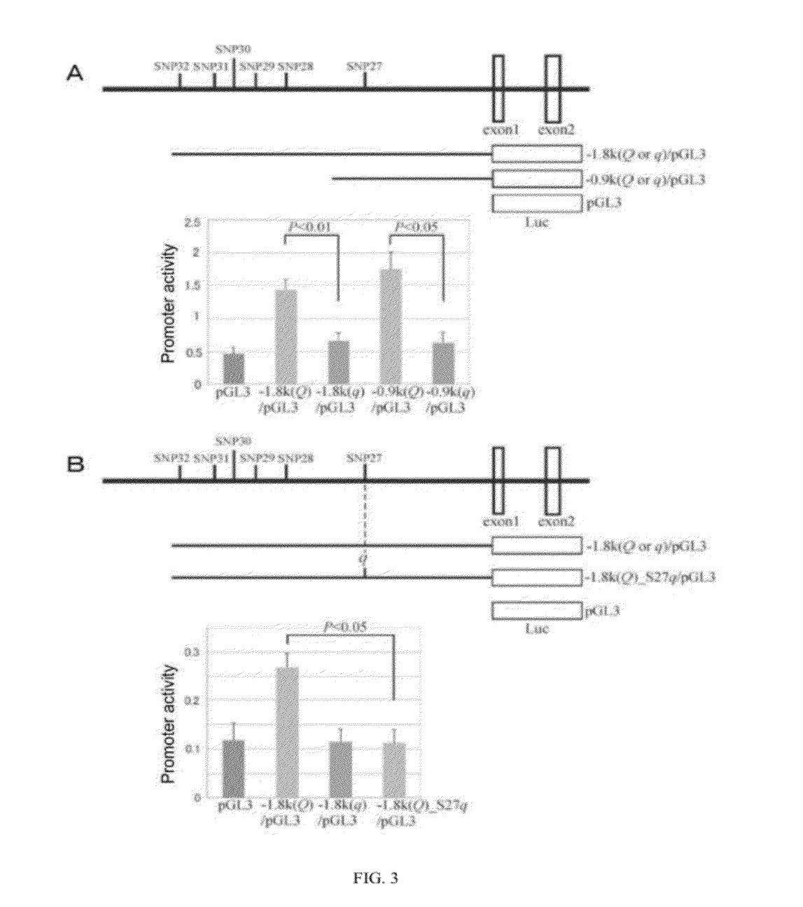

DNA sequences of the inserts of the obtained plasmids were confirmed by DNA sequencing. Each of the resulting recombinant plasmids, 1.8 kQ/pGL3, 1.8 kq/pGL3, 0.9 kQ/pGL3 and 0.9 kq/pGL3, was introduced into BIP cells and their luciferase activities were analyzed. The promoter activities in the q-types showed 54 or 63% reduction of the Q-types (FIG. 3A, P<0.05, N=4). Further, a new plasmid, −1.8 k(Q)_S27q/pGL3, that replaced the Q-type nucleotide at SNP27 of 1.8 kQ/pGL3 with the q-type nucleotide was prepared by PCR amplification of the entire construct of 1.8 kQ/pGL3 using a pair of the primers (SEQ ID NOs:84 and 85, shown below) containing the q-type SNP followed by sub-cloning of the 1.8 kb fragment into the multicloning site of pGL3-Basic. DNA sequence of the insert of the obtained plasmid was confirmed by DNA sequencing. As a result, the promoter activity of −1.8 k(Q)_S27q/pGL3 was reduced by 59% (FIG. 3B, P<0.05, N=3).

(SEQ ID NO: 84)
primer-q-f: AGCCTGGTAGGCTGCAGTCCAT
(SEQ ID NO: 85)
primer-q-r: CCTACCAGGCTCCTCCGTCCATGGGATTTT

The reduced level of promoter activity was comparable to that of the q-type construct in which the 1.8 kb upstream region of exon 1 containing SNP27 to SNP32 was inserted into the upstream of the luciferase reporter gene. These results indicate that SNP27 is responsible for the promoter activity of bovine pantophysin gene and therefore responsible for the marbling QTL.

In order to confirm that SNP27 is useful to evaluate genetic potential for marbling, correlations between breeding values of bulls and SNP27 were analyzed. As used herein, the “breeding value” refers to an estimated value of a genetic potential of a bull with regard to economic traits, and is calculated for each of the economic traits of carcass weight, rib-eye area, rib thickness, subcutaneous adipose thickness, yield and marbling, based on the scoring of offspring calves. In the present Example, the breeding value obtained by correcting the marbling (BMS) score, which concerns the degree of marbling and is registered between 1 to 12 from inferior to superior, was analyzed.

Analyses of 102 bulls in Hyogo prefecture whose breeding values were calculated from 20 or more animals of their progeny (data in 2007 to 2008, http://www.yumenet.tv/tajimausi/) revealed that the breeding values of Q-homozygotes were significantly higher than those of q-homozygotes (P=0.0032, Table 6). Also in 44 bulls kept in the Livestock Improvement Association of Japan whose breeding values were calculated from 50 or more animals of their progeny (analyzed from the data collected until 2008; “Evaluated values of bulls from the Project for Evaluation of Beef Cow and Breeding Cattle” http://liaj.lin.gr.jp/), the breeding values of Q-homozygotes were significantly higher than the breeding values of q-homozygotes (P=0.025, Table 7).

TABLE 6

| | | | | p value | p value | p value |
|---|---|---|---|---|---|---|
| | Animal | Breeding value (BMS) | | (t test) | (t test) | (t test) |
| SNP27 | number | Average | SD | AA/GG | AA/AG | AG/GG |
| AA | 38 | 1.08 | ±0.36 | 0.0032 | 0.097 | 0.070 |
| AG | 47 | 0.92 | ±0.47 | | | |
| GG | 17 | 0.66 | ±0.51 | | | |

TABLE 7

| | | | | p value | p value | p value |
|---|---|---|---|---|---|---|
| | Animal | Breeding value (BMS) | | (t test) | (t test) | (t test) |
| SNP27 | number | Average | SD | AA/GG | AA/AG | AG/GG |
| AA | 14 | 2.61 | ±0.74 | 0.025 | 0.096 | 0.55 |
| AG | 22 | 2.06 | ±1.13 | | | |
| GG | 22 | 1.87 | ±1.00 | | | |

In a further analysis of 224 animals of offspring from the Japanese Black bull C in Hyogo prefecture, it was demonstrated that the breeding values of Q-homozygotes were significantly higher than those of q-homozygotes (P=0.00021, Table 8), as well as that the breeding values of Q-homozygotes were also significantly higher than those of Qq heterozygotes (P=0.0026, Table 8).

TABLE 8

| | | | | p value | p value | p value |
|---|---|---|---|---|---|---|
| | Animal | Breeding value (BMS) | | (t test) | (t test) | (t test) |
| SNP27 | number | Average | SD | AA/GG | AA/AG | AG/GG |
| AA | 84 | 1.63 | ±0.17 | 0.00021 | 0.0026 | 0.21 |
| AG | 108 | 1.44 | ±0.19 | | | |
| GG | 32 | 1.35 | ±0.09 | | | |

In all of the 102 bulls in Hyogo prefecture (Table 6), the 58 bulls kept in the Livestock Improvement Association of Japan (Table 7) and the Japanese Black bull C offspring in Hyogo prefecture (Table 8), the breeding values of Q/q heterozygotes were higher than those of q-homozygotes.

Correlation between the number of the Q alleles at SNP27 and the breeding values in the 224 animals of offspring from Japanese Black bull C in Hyogo prefecture was examined by a regression analysis taking additive effect and dominance effect as the explanatory variables, and the effect of Q allele was found to be additive (p=0.0016).

Based on these results, it was concluded that SNP27 at position 49100585 on chromosome 4 of the bovine genomic sequence (Btau4.0) is useful as a marker to evaluate genetic potential for marbling and that the genetic potential for marbling can be evaluated by using this marker.

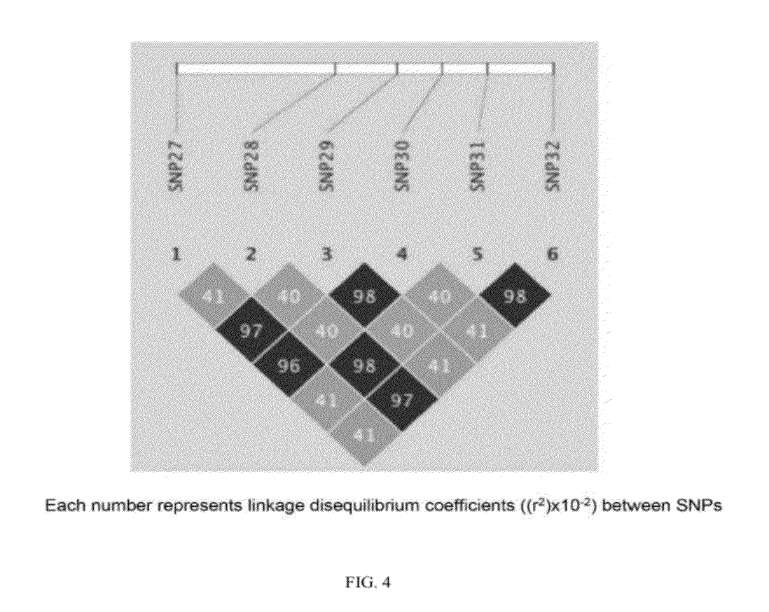

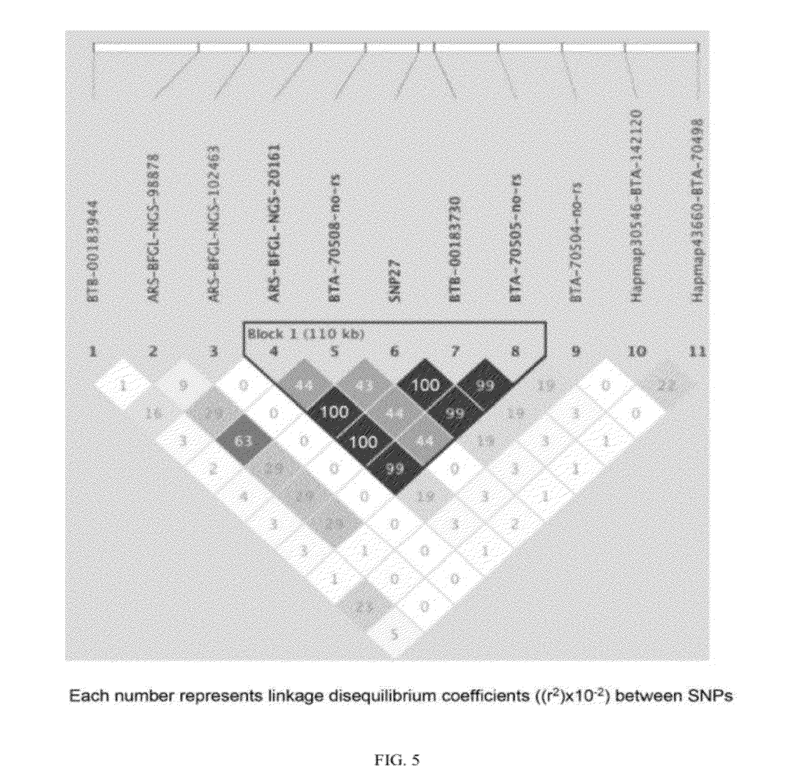

In addition, degrees of linkage disequilibrium from SNP27 to SNP32 in the population of Japanese Black used in the analysis shown in FIG. 1 were calculated by the same method as in FIG. 1B, and SNP29 (coefficient of linkage disequilibrium (r²)=0.97) and SNP30 (r²=0.96) were shown to be in linkage disequilibrium with SNP27 (FIG. 4). Another population of Japanese Black (568 animals, less than 10 half-siblings) was analyzed using Bovine SNP50 BeadChip (Illumina Inc.) to calculate degrees of linkage disequilibrium between SNP27 and other known SNPs located around the pantophysin gene, and each of ARS-BFGL-NGS-20161 (coefficient of linkage disequilibrium (r²)=1.000), BTB-00183730 (r²=1.000) and BTA-70505-no-rs (r²=0.996) was shown to be in linkage disequilibrium with SNP27 (FIG. 5). Accordingly, SNP29 and SNP30, as well as ARS-BFGL-NGS-20161, BTB-00183730 and BTA-70505-no-rs are also useful as markers to evaluate genetic potential for marbling of a bovine individual.

1. An isolated DNA selected from a group consisting of the following DNAs:
(1) DNA comprising a nucleotide corresponding to the nucleotide at 718^{th} position upstream of exon 1 of the bovine pantophysin gene as set forth in SEQ ID NO:1, wherein the nucleotide comprised in the DNA is A;
(2) DNA comprising a nucleotide corresponding to the nucleotide at 1322^{th} position upstream of exon 1 of the bovine pantophysin gene as set forth in SEQ ID NO:1, wherein the nucleotide comprised in the DNA is G; and
(3) DNA comprising a nucleotide corresponding to the nucleotide at the 1450^{th} position upstream of exon 1 of bovine pantophysin gene as set forth in SEQ ID NO:1, wherein the nucleotide comprised in the DNA is T.

2. A marker for evaluating a genetic potential for increasing the degree of marbling of a bovine individual, comprising a DNA having a nucleotide selected from a group consisting of the nucleotides at:
the 718^{th}, 1322^{th} and 1450^{th} position upstream of exon 1 of the bovine pantophysin gene as set forth in SEQ ID NO:1;
the 9847^{th} position upstream of exon 1 of the bovine pantophysin gene as set forth in SEQ ID NO:3;
the 47632^{th} position upstream of exon 1 of the bovine pantophysin gene as set forth in SEQ ID NO:4; and
the 27348^{th} position downstream of exon 7 of the bovine pantophysin gene as set forth in SEQ ID NO:2.

3. A method for evaluating a genetic potential for increasing the degree of marbling of a bovine individual, comprising a step selected from a group consisting of the following steps:
(1) judging whether or not a nucleotide corresponding to the 718^{th} nucleotide upstream of exon 1 of the bovine pantophysin gene as set forth in SEQ ID NO:1 is A, in at least one of alleles of DNA isolated from the bovine individual;
(2) judging whether or not a nucleotide corresponding to the 1322^{th} nucleotide upstream of exon 1 of the bovine pantophysin gene as set forth in SEQ ID NO:1 is G, in at least one of alleles of DNA isolated from the bovine individual;
(3) judging whether or not a nucleotide corresponding to the 1450^{th} nucleotide upstream of exon 1 of the bovine pantophysin gene as set forth in SEQ ID NO:1 is T, in at least one of alleles of DNA isolated from the bovine individual;
(4) judging whether or not a nucleotide corresponding to the 27348^{th} nucleotide downstream of exon 7 of the bovine pantophysin gene as set forth in SEQ ID NO:2 is A, in at least one of alleles of DNA isolated from the bovine individual;
(5) judging whether or not a nucleotide corresponding to the 9847^{th} nucleotide upstream of exon 7 of the bovine pantophysin gene as set forth in SEQ ID NO:3 is C, in at least one of alleles of DNA isolated from the bovine individual; and
(6) judging whether or not a nucleotide corresponding to the 47632^{th} nucleotide upstream of exon 1 of the bovine pantophysin gene as set forth in SEQ ID NO:4 is T, in at least one of alleles of DNA isolated from the bovine individual.

4. The method of evaluation according to claim 3, further comprising the step of
judging that the genetic potential for increasing the degree of marbling of the bovine individual is higher than genetic potential of a second bovine individual, wherein the nucleotide corresponding to the 718^{th} nucleotide upstream of exon 1 of the bovine pantophysin gene as set forth in SEQ ID NO:1 is G in the second bovine individual.

5. A method for selecting a bovine individual having a high genetic potential for increasing the degree of marbling, comprising the step of evaluating the genetic potential of the bovine individual by the method of evaluation according to claim 3.

6. The method according to claim 5, further comprising the step of selecting a bovine individual, wherein the bovine individual has been judged to be high in the genetic potential by the step of evaluation.


SEQUENCE LISTING (85 sequences)

SEQ ID NO: 1 - DNA, 2000 bp, Bos taurus
aagagagaacttaatactcaaggcacattctcttagcagcaatggcttcctgaagagaaacaaccatgccttccattagtgggcatgaaccagaagcagaagagaaagcaaaaaaaaaaaaaaaggcagaagagaggaaatagaaagttaaacttctcagaggctgagaaagggccatgtcaattccagttgctgaaaccttccaaaaattggggaggggaatacagaaaagccaaactggcttactatattacagtatattttttatttacatgaacaaataaagctgcaaagtgagcatcattctgacttgtcagtgtgtatctgatttggaaataacatgagagtctaatctgaatctttatgaatatgaagcccaagccagatagcactactagccacaacccaactccagggcaagcccccatacctcaacagagtggaagtaaaagtttagaaaacaaactaaagagaatacaacgagaagagtagcaaggaagccagtacggagatcactaccatgggaatcccataactgggtgccagaaaaatttttaaaagggggtggggagcaggaggacctgaggtattccttacagtggtctcaggagtactgggtactaatgtatggtcaccttccactgattctgctctaaggagcatctctgaagccttcttctttggtaagaggaatctgcactcattcactaggtatccactgatacaaggcactgtgcagggattgaagaatggaagtcacaatcaaaagaataagattgtctctgtggtccgggaaattgaaatatgctctctgcattctctgggaaacaaacaaggaggtaaacaggtctcaaatgagaaagcgttagcttcttagaaagaaggtcagtaacacttgagtaacttcaatggcgaggaacctgctgactcatagataagtgcttggaaaatcagtatacataggtaaatacttggaagctggtatattgagattccattgtgctagattcctttcttccgtattaggggtacatgatgtaagtccccttaccatacactccaaaggccagcatttcatctccaagaacttccaagatactagggaagccaaaaagatcatttcttcggggctccgataactggatgccagaaggtaggtgttacctaggcgactctcaaatgggtagtagcaattagccttctgtagagactccaagcggagaaggcaatggtaccccactccagtactctcgcctggaaaatcccatggacggaggagcctggtaggctgcagtccatggggtcgctgagggtcggacccgactgagcgacttccctttcacttttcactttcatgcattggagaaggcaatggcaacccactccagtgttcttgcctggagaatcccagggacgggggagcctggtggactgccgtctatggagtcgcacagagtcggacacgactgaagcgacttagccgcagcagcagcagagactccaagagcgccccacgtagaaaacttgactgacagggtaggagaggtgggtaggtgggttagaagagcctatcggaccaagtctttttctgttaggtataacgttttttgtaaatcgttcaaccctgactccaatggtaagtttgaaaataaacaatttccgtaagaatgatcaatcacgttcctgagagagaccctgttacagcgtggtcgctcaaagtccgttgttctgcagtaactttccatcattttaataaaaggtctcttaacaccagccttgaatcgcgccttcatttgtgtcaatgttataaacctagctaaaacattgggaatagtgacgggaagccagtgtaataaagctgctgggccaaagccgtaggtttccgctggatggttttcgcagcgcgaggcagctgtcccagctacgctggcgtgggactgagcgcgcgcatgcgcggcaggaatccgagcgcggaggctggacttgagggaagggggcgc

SEQ ID NO: 2 - DNA, 1000 bp, Bos taurus
tataagcaaagaggctcagagttgtggagtagaaccttcctctggcaatggggatactaggttctgccttcccagagcaggagtgaagctgtcactgccccaaagcaggaatgggctgccgccttgggtaaaggattggaggcctgggcactggagccctcctggtgccagccaggacccagaagtgcccaaggttgcacagtccctaaacctttccactataatgctacagtaggggagtaagagagcttctgggtgctgtaagagggcttcgggcaaaggcaatgagaaggaagaaagaagaatcataaggaaagcataggccaggaggaaagaggggcggacacggaatacatgaggggtgtggcaggtctctccaagacagctcgaaacaggcctgcacagccagcctcagcagctctctgcttcgaggaagcaccattagccaacagctaagcctaaacctcattctgatccctcacctgaacctcttcccaaaagtgaagggatgttcgaacagcctacctggaacatagatgggtggtttgtgtccctcccttgaaagatctcttttctcagccacaggaaaacaagtttcaaagtggattgggaaagaggggtggggtgcagatatggaaagaatgaggagggaacagatggtgcctgctaagacaaccagatttttccccccagttaatctgggaatgacctaatttacatagtatttgcataaatttccatttcaaattatagctgtcctaacttcaagtatacaggagaaaataaatgagatcaggcaatctactgtaattgaactcaagctgacttctgtgaaaggcaggtcaagctttcaaagtctggcagatgagaggaaggaattttgtggctgtccattagcaaaagggggcctggagaaagcctctccacctctcaaattaggcaaagttaccatgggtccatgtcgggtcagagatgtggggagatgaggaaattcaacctc

SEQ ID NO: 3 - DNA, 1000 bp, Bos taurus
tatgaacattctttcacacctctcctgctgtatatctgcttgagtttcccttgggtccatcccacagagtgaattaattgttgggtcatatgtatatgaatattcaactttacaagataatgccaaagtgttttccaaattagttacacaaatttgttacgttctataacaccaagagtgttctctgaaattcactttttatagtcccccttgttccgataactgccaaaaataagaattgctggttgacaaggaaatatttcatatctcctctgagtatttgcatttttaaagagaatgcttggagaggcagtgttttaatccaaagcaatggatttctctgactaaatatttcagggcctctatctctacatgctatatcagatttactcattcaatgcttcactggaatgcttgaattctgcagctgtgtgaggcctgaagaccaactagtgggacctgccttcgaacatcttaatgtctgctgtgtgagaaaaataagtaggctaattatattggacttggatgccatcttggtcttcctatctcttcatcacgttttcccaggtaagtaaggtatttctgattgacatttcagtcttaattaccagaccattagggaagtcccttgaaaatatatttgtttactattttttctttcacttatctttccttttagaatgtaaactctctgaggacaagaatatcttattccactttatatgactaaaatttaccaaagtacctggtccatacaggtaatcaataaatactggaaggaacaggtctgtcttcatgcctctctttcttctcaaatgattggcaaacatcgaaattaaatatgctaagaaaattcagtcttccctaaactaaactgtgaaggaatccaagtttgttttatttctgctttcccattttccttttcttcaacactgaccagcttctccccatacttcccaggcattcagaatcccttttgcatcttcttggcagccaggaa

SEQ ID NO: 4 - DNA, 1000 bp, Bos taurus
aaattctacgtttcttggcctttcttcaccaaactgctaaagcaaattgttttccagaaccgctcagaagagaagatgccagagagtggtttcaaagagtttctaatgaaaacgagctgtatttattctgacctccatgtgaaatggagttggtctgttgggttttttgttttgtcttcacttttctcatatttaagacttctcctttctgatgaagagttaaatgggatgactgaaagccactctgatagaaactgctgtattttccacttcgtataaagtacttcacacatggttaagcattccaacttaatggtatataacttcggtctcatcagtaaacactcatctattcattcattcagacacgtactaggtacctgctgggtatgggagatacaaacctaaatgatatatggtccctgccttgagcccacagtctaaaggtctaggctgaagatctagacttggaaggcagagcaagcaggtatccaccactgtttatgggagtccctggtggctcagttggtaaagaatctgcctgcaatgcaggagacccaggttcaatcccagtgttgggaagatcccctggagaagggaatggctacgcattccagtattcttgcctagagaattccatggacagaggagcctggcagactacagtccacgggattacaaagagtcagacatgactgagcaactaacactttccatttcatttcatgccatcactccaggctgtgtgtaggccaaaaagctggaaaaggctggaacctgaaggaaggccaacatttaggaagacagagacaacagcagggtgagagaggaaatcagagtatggtgaggcgcaggggttatgtcatggaagccaaaagagctgtttctaggagggaaaatgcaaaaatgtgacaatgcgcaaatgctgtgtggaggcccagtgagagagacctgaaagagttgttgggattccaagtttgctgatggccttggtagaggt

SEQ ID NO: 5 - DNA, 18 bp, Bos taurus
ccttgaattggagcagca

SEQ ID NO: 6 - DNA, 21 bp, Bos taurus
gccataggtagtgatgcaagc

SEQ ID NO: 7 - DNA, 21 bp, Bos taurus
gcagtagcctgttttctgctt

SEQ ID NO: 8 - DNA, 20 bp, Bos taurus
tatatgcacgttggccactc

SEQ ID NO: 9 - DNA, 20 bp, Bos taurus
aagaattcccatccctgagc

SEQ ID NO: 10 - DNA, 20 bp, Bos taurus
caactttctgcatgggagac

SEQ ID NO: 11 - DNA, 21 bp, Bos taurus
ggtggatcagcatcctgtaga

SEQ ID NO: 12 - DNA, 20 bp, Bos taurus
cttctctggccacccctatt

SEQ ID NO: 13 - DNA, 20 bp, Bos taurus
tccctcagacactcccttgt

SEQ ID NO: 14 - DNA, 20 bp, Bos taurus
agaggccaccattgagagaa

SEQ ID NO: 15 - DNA, 20 bp, Bos taurus
tcctcagtctgggaaaggaa

SEQ ID NO: 16 - DNA, 20 bp, Bos taurus
gcacacccaatgttaagctg

SEQ ID NO: 17 - DNA, 20 bp, Bos taurus
cattcttcccctcaaccatc

SEQ ID NO: 18 - DNA, 20 bp, Bos taurus
ggacactttttcttgcctga

SEQ ID NO: 19 - DNA, 20 bp, Bos taurus
atgtgcagggaaatgagcac

SEQ ID NO: 20 - DNA, 20 bp, Bos taurus
cgcctccaaaactgaatgag

SEQ ID NO: 21 - DNA, 20 bp, Bos taurus
agagagcctgggagttgtca

SEQ ID NO: 22 - DNA, 20 bp, Bos taurus
tatgggacctggatgcattg

SEQ ID NO: 23 - DNA, 21 bp, Bos taurus
tttgaatgagcatttctcacg

SEQ ID NO: 24 - DNA, 21 bp, Bos taurus
ctcaggcacgtggtatatgaa

SEQ ID NO: 25 - DNA, 20 bp, Bos taurus
tccagttctcccatgaaacc

SEQ ID NO: 26 - DNA, 20 bp, Bos taurus
aagaagagccctaacgcaga

SEQ ID NO: 27 - DNA, 21 bp, Bos taurus
ttggaagggctatagttgctg

SEQ ID NO: 28 - DNA, 20 bp, Bos taurus
ggaggcaggtggaatgttta

SEQ ID NO: 29 - DNA, 20 bp, Bos taurus
cagtggcaaaagctcagaac

SEQ ID NO: 30 - DNA, 20 bp, Bos taurus
aatgtggctgagaagccttg

SEQ ID NO: 31 - DNA, 20 bp, Bos taurus
gacctcattcaggaggcaaa

SEQ ID NO: 32 - DNA, 19 bp, Bos taurus
ggcaggacccttgaaattg

SEQ ID NO: 33 - DNA, 20 bp, Bos taurus
tttgaattcccaccttgaac

SEQ ID NO: 34 - DNA, 20 bp, Bos taurus
ttcccaaaagtccttgcatc

SEQ ID NO: 35 - DNA, 20 bp, Bos taurus
tcagactcagccaacagcac

SEQ ID NO: 36 - DNA, 20 bp, Bos taurus
actgctccacagcaaaaacc

SEQ ID NO: 37 - DNA, 20 bp, Bos taurus
ggtgaacagcagggaagttt

SEQ ID NO: 38 - DNA, 20 bp, Bos taurus
tgagctgtgcagtgtccaat

SEQ ID NO: 39 - DNA, 19 bp, Bos taurus
ctggaagtgtccgggaaat

SEQ ID NO: 40 - DNA, 20 bp, Bos taurus
tgcactcctaggtgaagacg

SEQ ID NO: 41 - DNA, 20 bp, Bos taurus
ccctaaaccctgtcaccaga

SEQ ID NO: 42 - DNA, 20 bp, Bos taurus
tcaggaatggaccagaggac

SEQ ID NO: 43 - DNA, 20 bp, Bos taurus
tctccagaacatgcatcctg

SEQ ID NO: 44 - DNA, 20 bp, Bos taurus
ggtggatcctggaattgcta

SEQ ID NO: 45 - DNA, 20 bp, Bos taurus
ttaaccagcaaggaggcaac

SEQ ID NO: 46 - DNA, 20 bp, Bos taurus
cggagcctaccccaaatagt

SEQ ID NO: 47 - DNA, 20 bp, Bos taurus
tggaggttttaagggcaaaa

SEQ ID NO: 48 - DNA, 20 bp, Bos taurus
cagataggcactggtccaca

SEQ ID NO: 49 - DNA, 20 bp, Bos taurus
cttttgccccacatgatttt

SEQ ID NO: 50 - DNA, 20 bp, Bos taurus
tgggtttagacgcagctctt

SEQ ID NO: 51 - DNA, 20 bp, Bos taurus
aaaccaggttcgagttgcac

SEQ ID NO: 52 - DNA, 20 bp, Bos taurus
acggtcgaaaatccagtcag

SEQ ID NO: 53 - DNA, 20 bp, Bos taurus
tctctcctccttcccattca

SEQ ID NO: 54 - DNA, 20 bp, Bos taurus
cttctgctggccactctttc

SEQ ID NO: 55 - DNA, 20 bp, Bos taurus
cagtggctgcactaatccaa

SEQ ID NO: 56 - DNA, 20 bp, Bos taurus
cctcagaactgccctgaaag

SEQ ID NO: 57 - DNA, 20 bp, Bos taurus
caaaggccagcatttcatct

SEQ ID NO: 58 - DNA, 20 bp, Bos taurus
ccacctctcctaccctgtca

SEQ ID NO: 59 - DNA, 20 bp, Bos taurus
gcagggattgaagaatggaa

SEQ ID NO: 60 - DNA, 20 bp, Bos taurus
tggcctttggagtgtatggt

SEQ ID NO: 61 - DNA, 20 bp, Bos taurus
ggaagccagtacggagatca

SEQ ID NO: 62 - DNA, 20 bp, Bos taurus
ccggaccacagagacaatct

SEQ ID NO: 63 - DNA, 20 bp, Bos taurus
aattggggaggggaatacag

SEQ ID NO: 64 - DNA, 20 bp, Bos taurus
tctccgtactggcttccttg

SEQ ID NO: 65 - DNA, 20 bp, Bos taurus
ctgagaaagggccatgtcaa

SEQ ID NO: 66 - DNA, 20 bp, Bos taurus
ctggcttgggcttcatattc

SEQ ID NO: 67 - DNA, 22 bp, Bos taurus
acaggcgctgcacaccacgagg

SEQ ID NO: 68 - DNA, 27 bp, Bos taurus
ctcggtctattctgcgtgccacttgga

SEQ ID NO: 69 - DNA, 26 bp, Bos taurus
ccaggctgaagtgctcaccaaccaca

SEQ ID NO: 70 - DNA, 25 bp, Bos taurus
cgctcctatgccagcagtctcttgg

SEQ ID NO: 71 - DNA, 27 bp, Bos taurus
gggaggttatgaggcttaataaagaag

SEQ ID NO: 72 - DNA, 19 bp, Bos taurus
ggaagacctgcggcttgac

SEQ ID NO: 73 - DNA, 27 bp, Bos taurus
actactgtcaacagctatttcatgatc

SEQ ID NO: 74 - DNA, 26 bp, Bos taurus
catgatgggattttgtcttaattcgc

SEQ ID NO: 75 - DNA, 24 bp, Bos taurus
cttctgctgtatgttggttacacg

SEQ ID NO: 76 - DNA, 26 bp, Bos taurus
ggtagctgctttaatgtctgtaagag

SEQ ID NO: 77 - DNA, 25 bp, Bos taurus
ttacatgattttggctacagaggag

SEQ ID NO: 78 - DNA, 25 bp, Bos taurus
ttcctttgaagttaaccaaatgagc

SEQ ID NO: 79 - DNA, 1788 bp, Bos taurus
attggggaggggaatacagaaaagccaaactggcttactatattacagtatattttttatttacatgaacaaataaagctgcaaagtgagcatcattctgacttgtcagtgtgtatctgatttggaaataacatgagagtctaatctgaatctttatgaatatgaagcccaagccagatagcactactagccacaacccaactccagggcaagcccccatacctcaacagagtggaagtaaaagtttagaaaacaaactaaagagaatacaacgagaagagtagcaaggaagccagtacggagatcactaccatgggaatcccataactgggtgccagaaaaatttttaaaagggggtggggagcaggaggacctgaggtattccttacagtggtctcaggagtactgggtactaatgtatggtcaccttccactgattctgctctaaggagcatctctgaagccttcttctttggtaagaggaatctgcactcattcactaggtatccactgatacaaggcactgtgcagggattgaagaatggaagtcacaatcaaaagaataagattgtctctgtggtccgggaaattgaaatatgctctctgcattctctgggaaacaaacaaggaggtaaacaggtctcaaatgagaaagcgttagcttcttagaaagaaggtcagtaacacttgagtaacttcaatggcgaggaacctgctgactcatagataagtgcttggaaaatcagtatacataggtaaatacttggaagctggtatattgagattccattgtgctagattcctttcttccgtattaggggtacatgatgtaagtccccttaccatacactccaaaggccagcatttcatctccaagaacttccaagatactagggaagccaaaaagatcatttcttcggggctccgataactggatgccagaaggtaggtgttacctaggcgactctcaaatgggtagtagcaattagccttctgtagagactccaagcggagaaggcaatggtaccccactccagtactctcgcctggaaaatcccatggacggaggagcctggtaggctgcagtccatggggtcgctgagggtcggacccgactgagcgacttccctttcacttttcactttcatgcattggagaaggcaatggcaacccactccagtgttcttgcctggagaatcccagggacgggggagcctggtggactgccgtctatggagtcgcacagagtcggacacgactgaagcgacttagccgcagcagcagcagagactccaagagcgccccacgtagaaaacttgactgacagggtaggagaggtgggtaggtgggttagaagagcctatcggaccaagtctttttctgttaggtataacgttttttgtaaatcgttcaaccctgactccaatggtaagtttgaaaataaacaatttccgtaagaatgatcaatcacgttcctgagagagaccctgttacagcgtggtcgctcaaagtccgttgttctgcagtaactttccatcattttaataaaaggtctcttaacaccagccttgaatcgcgccttcatttgtgtcaatgttataaacctagctaaaacattgggaatagtgacgggaagccagtgtaataaagctgctgggccaaagccgtaggtttccgctggatggttttcgcagcgcgaggcagctgtcccagctacgctggcgtgggactgagcgcgcgcatgcgcggcaggaatccgagcgcggaggctggacttgagggaagggg

SEQ ID NO: 80 - DNA, 935 bp, Bos taurus
aaaggccagcatttcatctccaagaacttccaagatactagggaagccaaaaagatcatttcttcggggctccgataactggatgccagaaggtaggtgttacctaggcgactctcaaatgggtagtagcaattagccttctgtagagactccaagcggagaaggcaatggtaccccactccagtactctcgcctggaaaatcccatggacggaggagcctggtaggctgcagtccatggggtcgctgagggtcggacccgactgagcgacttccctttcacttttcactttcatgcattggagaaggcaatggcaacccactccagtgttcttgcctggagaatcccagggacgggggagcctggtggactgccgtctatggagtcgcacagagtcggacacgactgaagcgacttagccgcagcagcagcagagactccaagagcgccccacgtagaaaacttgactgacagggtaggagaggtgggtaggtgggttagaagagcctatcggaccaagtctttttctgttaggtataacgttttttgtaaatcgttcaaccctgactccaatggtaagtttgaaaataaacaatttccgtaagaatgatcaatcacgttcctgagagagaccctgttacagcgtggtcgctcaaagtccgttgttctgcagtaactttccatcattttaataaaaggtctcttaacaccagccttgaatcgcgccttcatttgtgtcaatgttataaacctagctaaaacattgggaatagtgacgggaagccagtgtaataaagctgctgggccaaagccgtaggtttccgctggatggttttcgcagcgcgaggcagctgtcccagctacgctggcgtgggactgagcgcgcgcatgcgcggcaggaatccgagcgcggaggctggacttgagggaagggg

SEQ ID NO: 81 - DNA, 29 bp, Bos taurus
ggggctagcaattggggaggggaatacag

SEQ ID NO: 82 - DNA, 28 bp, Bos taurus
gggagatctccccttccctcaagtccag

SEQ ID NO: 83 - DNA, 29 bp, Bos taurus
ggggctagccaaaggccagcatttcatct

SEQ ID NO: 84 - DNA, 22 bp, Bos taurus
agcctggtaggctgcagtccat

SEQ ID NO: 85 - DNA, 30 bp, Bos taurus
cctaccaggctcctccgtccatgggatttt